(19) United States
(12) Patent Application Publication
(10) Pub. No.: US 2015/0290401 A1
(43) Pub. Date: Oct. 15, 2015

(54) PROTECTIVE INJECTION AID

(71) Applicant: THE TRUSTEES OF THE UNIVERSITY OF PENNSYLVANIA, Philadelphia, PA (US)

(72) Inventors: Kellie Barnes, Philadelphia, PA (US); Elisa Hepp, Philadelpha, PA (US); Thomas P. Fitzsimons, Swarthmore, PA (US)

(21) Appl. No.: 14/443,761

(22) PCT Filed: Nov. 18, 2013
(86) PCT No.: PCT/US13/70516

§ 371 Date: May 19, 2015

Related U.S. Application Data
(60) Provisional application No. 61/728,758, filed on Nov. 20, 2012.
(60) Provisional application No. 61/831,332, filed on Jun. 5, 2013.

(51) Int. Cl. A61M 5/42; A61M 5/00

(52) U.S. Cl. CPC A61M 5/425; A61M 5/002

(57) ABSTRACT

The present aids and their attendant methods allow injectors to achieve injection and sampling using syringes with reduced danger of incurring accidental needle punctures upon themselves. Injection aids feature a needle cap receptacle as part of the aid such that a needle may be uncapped, the cap being retained on the injection aid. In accordance with present methods, a fold of skin may be created with the aid in order to facilitate insertion of the needle. The used needle may thereafter be inserted into the protective cap while that cap is retained on the injection aid, rather than being held in the fingers of the injector. Kits are also provided comprising the injection aids of the invention.

### RELATED APPLICATIONS

This application claims priority to U.S. Provisional Patent Application No. 61/728,758, entitled “Protective Injection Aid,” and filed Nov. 20, 2012, and U.S. Provisional Patent Application No. 61/831,332, entitled “Protective Injection Aid,” and filed Jun. 5, 2013, both of which are incorporated herein by reference in their entireties.

### TECHNICAL FIELD

The present invention is directed to devices for the facilitation of hypodermic subcutaneous, intravenous, and other injections into the bodies of mammals, especially humans. The invention provides improved stabilization of tissue to be injected together with enhanced protection for the person engaged in performing the injection.

### BACKGROUND

Inadvertent needle sticks are a significant problem in the health care delivery system causing anxiety, expense, and medical risk. The United States Occupational Safety and Health Administration has a policy on needle stick safety and reporting. See The Needle stick Safety and Prevention Act (the Act) (Pub. L. 106-430) enacted Nov. 6, 2000). The Unite Kingdom National Health Service, National Audit Office report of Apr. 2003, A Safer Place to Work—Improving the Management of Health and Safety Risks in NHS Trusts, found that needle stick and sharps injuries account for 17 per cent of accidents to NHS staff and are the second most common cause of injury.

While the overall incidence percentage of accidental needle sticks is small, the number of injections is very large. Accordingly, this is a significant economic and medical problem. Estimates are that there are over 600,000 needle sticks at a cost of $100-450 million in the US each year. Curr Med Res Opin. 2007 September; 23(9):2093-105; Costs of Needlestick Injuries and Subsequent Hepatitis and HIV Infection; Leigh J P, Gillen M, Franks P, Sutherland S, Nguyen H H, Steenland K, Xing G.

Major blood-borne pathogens of concern associated with needle stick injury include hepatitis B virus (HBV), hepatitis C virus (HCV), and human immunodeficiency virus (HIV). Other infectious agents also have the potential for transmission through needle stick injury. These include human T lymphotrophic retroviruses (HTLV I & II). hepatitis D virus (HDV or delta agent, which is activated in the presence of HBV) hepatitis G virus (GB virus or GBV-C), cytomegalovirus (CMV), Epstein Barr Virus (EBV), parvovirus B19, transfusion-transmitted virus (TTV), West Nile Virus (WNV), malarial parasites, and prion agents such as those associated with transmissible spongiform encephalopathies (TSE).

It will be understood by persons skilled in the art that it is conventional for hypodermic needles to be supplied with a protective cap prior to use. This cap maintains sterility and protects against accidental puncture. The needle is removed from the cap in preparation for injection, typically, by holding the cap in one hand while the needle is removed by the other. Injection is typically accomplished by manipulating the tissue to be injected with one hand of the injector while the other, holds the needle and performs the injection. Following injection, the cap is typically replaced by holding the cap in one hand while the used, contaminated needle is replaced into it by the other hand. More commonly and within current OSHA and FDA guidelines hypodermic needles with needle guards are used to protect the user against accidental needle sticks with contaminated needles (REF). Alternatively separate devices are used to keep the needle tip away from the user's fingers during the recapping process (REF). Despite these devices needle stick report rates are essentially unchanged, and roughly two thirds of needle sticks in a Massachusetts study occurred with devices designed to prevent them. http://www.safeincommon.org/needlestick-statistics; downloaded Nov. 2, 2012)

Needle sticks occur predominantly at two points in the use of syringes; when an injection is being delivered and when a needle used in an injection (or withdrawal of fluid) is being recapped prior to disposal. While needle sticks during recapping are generally considered more serious they must both be reported, documented and an appropriate treatment plan developed. Hence, it is important to provide protection at both points in the procedure. In both cases, one hand of the injector is necessarily in proximity to the uncapped needle. A number of devices have been proposed previously to minimize the problem, yet many hospitals and clinics have not adopted these solutions. There are a number of reasons for this. For example, Terumo Patent US20070179451—Safety needle assembly, demonstrates both current thinking on devices to cap needles at the end of a procedure, as well as detailed references to the prior art. Similarly, Patent U.S. Pat. No. 6,648,857 directed to Disposable Needle Stick Prevention Aid to Prevent Needle Stick Injury, demonstrates attempts to prevent needle sticks during recapping using a scoop technique. Design patent D340,113 shows a device intended to be used to allow intramuscular and subcutaneous injections while preventing inadvertent needle sticks while injecting, rather than recapping. U.S. Pat. No. 5,147,306 exemplifies devices using spring or other power for puckering skin with a clothespin—like assembly. None of these proposed approaches satisfies the need for an effective and convenient device for reducing the likelihood of needle sticks during and following injection.

### SUMMARY

The present invention provides devices, methods and kits for reducing the risk of needle sticks during and after medical injection. The invention is simple, inexpensive to implement and protects against inadvertent needle sticks at both ends of the procedure: injection and recapping. This is accomplished by providing devices which allow injection to be performed in manners which keep the injector's fingers remote from the needle tip during both the injection and recapping processes. This is accomplished by using a tool to pinch the skin during the injection process. This tool features a receptacle for the needle cap such that upon completion of the injection, the needle may be replaced into the needle cap while the cap is held on the device rather than in the fingers. The injector's fingers are kept away from the needle tip during recapping.

### BRIEF DESCRIPTION OF THE DRAWINGS

FIGS. 1, 2, 3, 4 and 5 show examples of the articles of the invention.

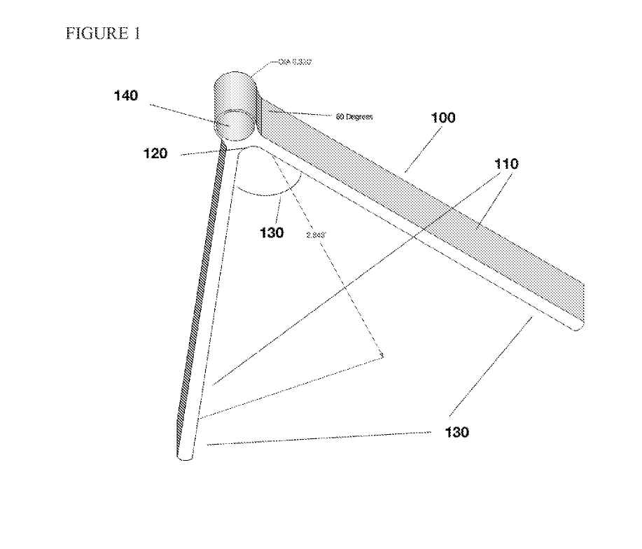

By reference to FIG. 1, Injection aid 100 is preferably shaped generally in the form of a third degree lever such as forceps or a pincer. The aid may be seen to comprise a pair of arms 110 angularly disposed and resiliently connected through a fulcrum 120. The arms are normally open to provide tips of the arms with a dimensional spread 130. The angle of the arms to each other is conveniently between about 10 and 80 degrees with angles between about 20 and 40 degrees being preferred. While it is preferred that the aids of the present invention be made of resilient material such that they may attain a normally open geometry until deformed by the injector, a resilient device or spring may also be employed for this purpose. The dimensions of the aid are selected to be consistent with the objectives of the invention. Accordingly, the aid is designed to be able to pinch a fold of tissue from a patient so that the injector may perform injection into the fold. Thus, the arms 110 are conveniently between about 5 to 8 inches in length with a spread 130 between about 1½ and 4 inches at the tips. The aid has a thickness and other dimensions which, taken with the material from which the aid is comprised, is convenient for holding in the hands of an injector with good control. Such materials must also be sterilizable in most cases. Such material may be metal, plastic or combinations thereof which is consistent with the operation of the aid. The aids may injection molded using nylon, ABS or any other thermoplastic known in the art. Similarly the aid can be stamped or machined from steel, aluminum or any other suitable metallic or non-metallic material so long as the material is sterilizable and resilient. It is believed that these qualitative design factors are easily understood by persons skilled in the art.

The aid 100 is provided with a receptacle 140 having dimensions which interact with the caps of at least some syringes such that the aid can hold a cap while the syringes are operated. It is preferred for some embodiments that the receptacle be tapered to as to provide a good friction or interference fit with syringe caps. The location of the receptacle may be any place on the aid which allows for use of the aid during injection without interference from a cap in place in the receptacle. Such location is also one which is convenient for holding a syringe cap while the syringe is uncapped to expose the needle. Further, the location is one which permits the recapping of the needle by the cap without the need to do so in proximity to the fingers of the injector.

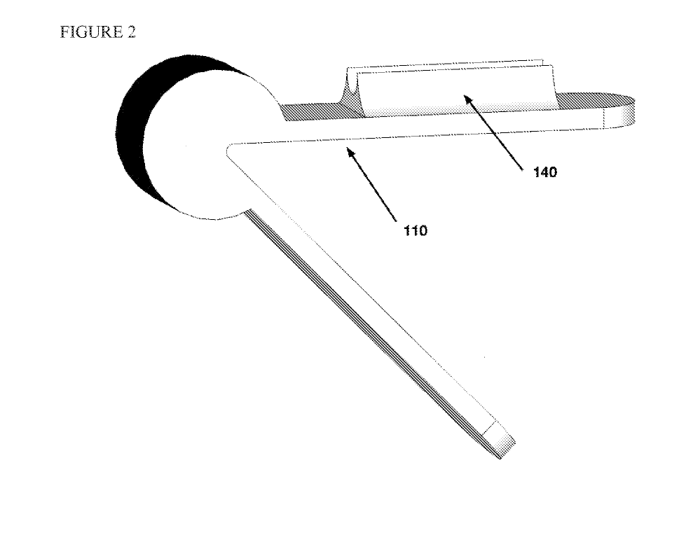

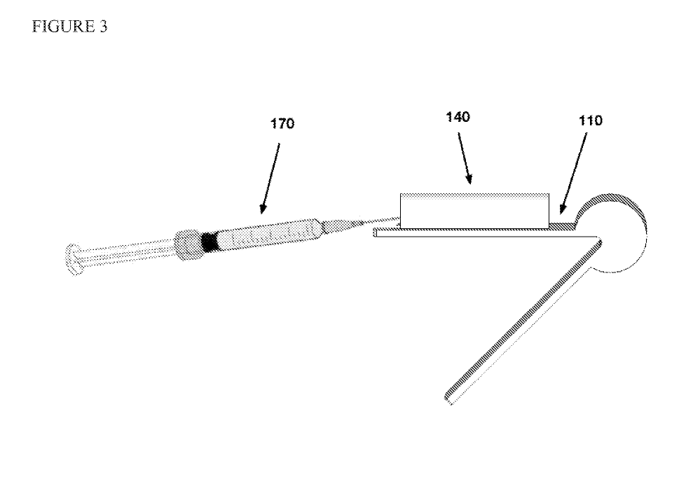

FIGS. 2, 3 and 4 depict alternative arrangement of elements for injection aids of the present invention. In FIG. 2, receptacle 140 is formed on one of the arms such that a syringe cap may be held in place generally collinearly with the arm such that the injection aid may become both pincher and cap. FIG. 3 shows the receptacle 140 on the outside aspect of an arm of the aid with a hypodermic syringe 170 at the entrance to the receptacle. Other arrangements may be arrived at following appreciation of the present description in view of the present objects to be attained. While it is preferred that the receptacle be integral with the overall aid, a receptacle may be separately shaped and attached to the aid for use. Thus the receptacle may be adhered, place with fasteners or otherwise associated with the aid. Even though the receptacle may, in some embodiments, be seen to be separate from the arms and fulcrum, it will be understood to form a part of the injection aids of this invention.

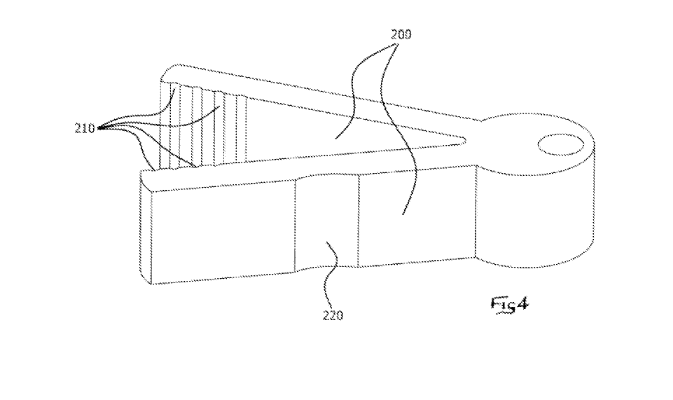

In FIG. 4, corrugations 210 are shown on the inside aspect 200 of the arms of the injection aid. At least portions of the arms at the location where skin is to be entrapped and pinched, are optionally made of a different material from the material of the rest of the arms of the aid. Placing the corrugations, including those elaborated with a different material on the inside of the arms 210 facilitate the holding or pinching of the tissue of a patient by the arms to maintain the tissue in a fold suitable for injection. Alternatively or additionally, corrugations or other patterning may be on the outside of the arms to prevent finger slippage on the part of the injector. Use of a relatively soft material such as a silicone or other rubber improves the holding power of the arms when engaged. Other features for improving the holding power of the arms are shown in FIG. 4. These include surface scribing, detents and roughening. Any of these and, indeed other ways of improving the holding power of arm surfaces in this context may be employed. As will be appreciated, the corrugations, scribing, detents or other features are located at least in part at the inwardly facing, outwardly facing or both surfaces of one or both arms. Detents 220 or other irregularities on the outwardly facing portions of the arms may be employed to facilitate finger actuation of the injection aids. As stated, it is preferred that a rubbery or other material having a higher effective coefficient of friction with mammalian skin be featured at the portions of the injection aid intended for contact with such skin. For purposes of this specification, the term “rubbery” will be used to connote all such embodiments. Further, a combination of rubbery material and corrugations or other features may be employed.

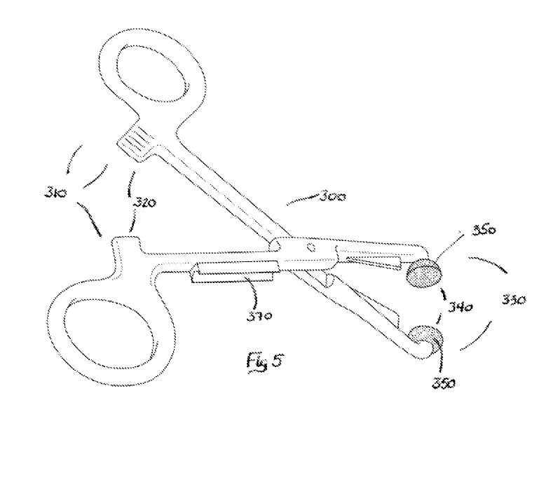

FIG. 5 discloses a type 1 lever arrangement 300 similar in overall configuration to a hemostat. Hand force is used to operate the injection aid through placement of fingers at the operator end 310 of the device. As is shown, a traditional hemostat locking mechanism 320 may be used, but this is entirely optional. The ends of the levers 330 remote from, distal to, the operator ends are preferably tipped with widened or enhanced tips 340. These tips 340, which are intended for holding the skin in a pinched position during insertion of a syringe needle, may also be furnished with corrugations, detents, scribing or other features and/or preferably have a rubbery gripping surface, 350.

As with certain other preferred embodiments, the injection aid of FIG. 5 features a receptacle 370 for holding the cap of a syringe during operation of the syringe. The cap may be replaced following the syringing operation by placing the syringe needle within the cap while it is retained on the injection aid. The covered needle may then be removed by unclipping the capped needle from the aid in a way which renders risk to the operator to be very low.

The devices of this invention are preferably hand actuated rather than spring operated or mechanically actuated. Unlike spring operated “clothespin” like devices of the prior art, the present hand actuated devices provide operator feedback such that effective pinching of skin may be performed without the use of excessive force. Patient pain, bruising and other tissue injury is, thus avoided. As will be appreciated, many patients, especially the elderly present with very delicate skin; skin susceptible to skin and even subcutaneous injury. Pain is easily caused by overzealous pinching of the skin.

It will be appreciated that the receptacle for protective syringe caps is preferably located at least somewhat remotely (distally) from the location where the operator's fingers will be located. In this regard, placing the receptacle sufficiently far from the place where the operator's fingers will apply the actuating force for operation of the injection aid is desirable. In so doing, needle sticks will be minimized.

In use, an injection aid of the present invention may be seen to provide enhanced protection for an injector during the injection of a patient. A professional injector, such as a nurse, doctor, phlebotomist, or aide attends a patient in need of injection, either for delivery of a medicament or for removal of a blood or tissue sample. A syringe having a protective cap is therefore present along with an injection aid of the present invention. In a preferred embodiment, the injector places the capped needle into the receptacle forming part of the present injection aid. The syringe is removed from the cap; the cap being retained within the receptacle. The cap may be removed either in a separate motion from the action of placing the cap within the receptacle or the two may form a single motion. The injection aid is then used to pinch or enclose a fold of skin on the patient sufficient for the injective procedure being performed. The needle is then injected into the fold of skin between the arms of the aid, operated, and removed. Upon removal, the used needle is inserted into the cap in place in the receptacle and the capped, used needle removed. Throughout the process, the fingers of the injector do not come into proximity with the uncapped needle.

While the present invention provides improved devices and methods for achieving injection in human patients, the same or similar devices and kit may also be used with non-human animals. Thus, for animals of different sizes, injection aids having different dimensions may find convenience and utility. For the purposes of this specification, both human and non-human animals may be referred to as patients.

The present invention also provides kits for use with the present injection aids and in furtherance of the present injections methods. A kit may be comprised of a container, such as a box, bag, case or other container together with at least one injection aid in accordance with the present invention. Additionally, instructions, preferably at least a portion of which are graphic, are preferably provided in order to instruct users in the operation of the injection aid. In preferred embodiments of the invention, the kits also include at least one further injection aid, syringe protective cap, syringe needle, syringe, syringe assembly, sterile skin wipe, pressure bandage, catheter, port or introducer. The kit may include a pre-filled syringe or a contained of injectable material such as a medicine for injection. The syringe may be a sheath for containing tubes for the collection of bodily fluids such as a Vacutainer® or a lancet. The components of the kits are preferably arranged within the container in an efficient way such that a user of the kit can easily access those components.

The kits of the present invention may also include a sharps container. Conventionally, needles and other things capable of puncturing the skin of a patient or operator are placed into a protective container, conventionally called a sharps container, immediately after use. The present kits may have a suitably protected location for such sharps such that used needles may be placed there for containment and/or transport until a more conventional sharps container may be accessed such as might be done in a long-term care facility. It is preferred that the sharps container of the kits, if included, be able to be inserted into a larger, conventional sharps container directly and without the necessity of handling syringes apart from the container. This is particularly useful for veterinary kits and for kits intended for use away from medical institutions.

What is claimed is:

1. An injection aid having a pair of angularly disposed arms connected through a fulcrum and further comprising a syringe cap receptacle.

2. The injection aid of claim 1 in the form of a type 1 or type 3 lever.

3. The injection aid of claim 1 formed from a sterilizable, resilient plastic.

4. The injection aid of claim 1 formed from sterilizable metal.

5. The injection aid of claim 1 wherein the receptacle is located distally from the location intended for placement of an operator's fingers.

6. The injection aid of claim 1 wherein the receptacle is located proximate the tip of one arm.

7. The injection aid of claim 1 wherein at least one surface of the arms intended for contact with patient skin is corrugated.

8. The injection aid of claim 1 wherein at least one surface of the arms intended for contact with patient skin has a rubbery surface.

9. A method for injecting a patient with a syringe having a needle covered by a cap comprising:
a. removing the cap;
b. placing the cap covered needle into the syringe cap receptacle of an injection aid having a pair of angularly disposed arms connected through a fulcrum and further comprising said syringe cap receptacle, said placement involving either the same or different motion from paragraph a hereof;
c. enclosing a fold of skin within the arms of the injection aid;
d. placing the needle within the fold of skin;
e. effecting operation of the syringe; and
f. placing the used needle into the cap held within the receptacle.

10. A kit comprising at least one injection aid having a pair of angularly disposed arms resiliently connected through a fulcrum, and further comprising a syringe cap receptacle, instructions for using the injection aid, and at least one further injection aid, syringe protective cap, syringe needle, syringe, syringe assembly, sterile skin wipe, pressure bandage, catheter, lancet, port, introducer, pre-filled syringe or a contained of injectable material such as a medicine for injection, a sheath for containing tubes for the collection of bodily fluids.

11. The kit of claim 10 further comprising a sharps container.
